(19) United States
(12) Patent Application Publication
(10) Pub. No.: US 2024/0081323 A1
(43) Pub. Date: Mar. 14, 2024

(54) ORGAN CARE SOLUTION FOR EX-VIVO MACHINE PERFUSION OF DONOR LUNGS

(71) Applicant: TransMedics, Inc., Andover, MA (US)

(72) Inventors: Waleed H. HASSANEIN, North Andover, MA (US); Ihab Abdel FATTAH, Andover, MA (US); Paul LEZBERG, Westford, MA (US); Tamer I. KHAYAL, North Andover, MA (US); Robert HAVENER, Lynnfield, MA (US); Anas ABDELAZIM, Cleveland, OH (US)

(21) Appl. No.: 18/512,184

(22) Filed: Nov. 17, 2023

Related U.S. Application Data
(63) Continuation of application No. 17/869,701, filed on Jul. 20, 2022, now Pat. No. 11,856,944.
Application No. 17/869,701 is a division of application No. 13/446,706, filed on Apr. 13, 2012, now abandoned.
(60) Provisional application No. 61/475,524, filed on Apr. 14, 2011.

(51) Int. Cl. A01N 1/02

(52) U.S. Cl. CPC A01N 1/0226; A01N 1/0247

(57) ABSTRACT

An ex-vivo lung solution for machine perfusion of donor lungs on OCS. The solution may be mixed with whole blood or packed red blood cells to form the OCS lung perfusion solution.

### CROSS-REFERENCE TO RELATED APPLICATIONS

This application is a continuation of U.S. patent application Ser. No. 17/869,701 filed on Jul. 20, 2022, which is a divisional application of U.S. patent application Ser. No. 13/446,706 filed on Apr. 13, 2012, which claims the benefit under 35 U.S.C. § 119(e), of provisional application U.S. Ser. No. 61/475,524, filed on Apr. 14, 2011, entitled, “ORGAN CARE SOLUTION FOR EX-VIVO MACHINE PERFUSION OF DONOR LUNGS”, the entire subject matter of which is incorporated herein by reference. This application also incorporates by reference, the entirety of U.S. application Ser. No. 12/099,715, filed on Apr. 8, 2008, entitled, “SYSTEMS AND METHODS FOR EX VIVO LUNG CARE”.

### TECHNICAL FIELD

The disclosure generally relates a perfusion solution for ex-vivo organ care. More particularly, the disclosure relates to a solution for machine perfusion of donor lungs on an organ care system (“OCS”) at physiologic or near-physiologic conditions.

### BACKGROUND

Current organ preservation techniques typically involve hypothermic storage of the organ in a chemical perfusion solution. In the case of the lung, it is typically flushed with a cold preservation solution such as Perfadex™ and then immersed in that same cold solution until it is transplanted. These techniques utilize a variety of cold preservation solutions, none of which sufficiently protect the lungs from tissue damage resulting from ischemia. Such injuries are particularly undesirable when an organ, such as a lung, is intended to be transplanted from a donor into a recipient.

Using conventional approaches, tissue injuries increase as a function of the length of time an organ is maintained ex-vivo. For example, in the case of a lung, typically it may be preserved ex-vivo for only about 6 to about 8 hours before it becomes unusable for transplantation. As a result, the number of recipients who can be reached from a given donor site is limited, thereby restricting the recipient pool for a harvested lung. Compounding the effects of cold ischemia, current cold preservation techniques preclude the ability to evaluate and assess an organ ex-vivo. Because of this, less-than-optimal organs may be transplanted, resulting in post-transplant organ dysfunction or other injuries, or resuscitatable organs may be turned down.

Prolonged and reliable ex-vivo organ care would also provide benefits outside the context of organ transplantation. For example, a patient's body, as a whole, can typically tolerate much lower levels of chemo-, bio- and radiation therapy than many particular organs. An ex-vivo organ care system would permit an organ to be removed from the body and treated in isolation, reducing the risk of damage to other parts of the body. Thus, there is a need to develop techniques and perfusion solutions that do not require hypothermic storage of the organ and extend the time during which an organ can be preserved in a healthy state ex-vivo. Such techniques would improve transplant outcomes and enlarge potential donor and recipient pools.

### SUMMARY

The disclosure provides improved methods, solutions, and systems related to ex-vivo organ care. In general, in one aspect, the disclosure features a lung OCS solution for machine perfusion of donor lungs on OCS at near physiologic conditions. In another aspect, the disclosure includes a system and method for perfusing one or more lungs ex-vivo for an extended period of time in a functional and viable state maintenance mode at near physiologic conditions. In another aspect the disclosure includes a method of producing a solution for ex-vivo perfusion of a donor lung at near physiologic conditions.

The present disclosure describes an OCS lung perfusion solution that can be used for machine perfusion of donor lungs on OCS. The solution may include energy-rich perfusion nutrients, as well as a supply of therapeutics, vasodilators, endothelial stabilizers, and/or preservatives for reducing edema and providing endothelial support to the lungs. In a preferred embodiment, the solution comprises: dextran 40; sodium chloride; potassium chloride; magnesium sulfate anhydrate; disodium phosphate anhydrate; monopotassium phosphate; glucose monohydrate; milrinone; nitroglycerin; insulin; a multi-vitamin (M.V.I. Adult® or equivalent); sodium bicarbonate; methylprednisolone (SoluMedrol® or equivalent); cefazolin; Ciprofloxacin; voriconazole. The solution is mixed with whole blood or packed red blood cells to form the OCS lung perfusion solution. The solution provides the components for maintaining a functional (e.g., under respiration) and viable lung ex-vivo at near physiologic conditions.

According to certain embodiments, solutions with particular solutes and concentrations are selected and proportioned to provide for the organ to function at physiologic or near physiologic conditions. For example, such conditions include maintaining organ function at or near a physiological temperature and/or preserving an organ in a state that permits normal cellular metabolism, such as protein synthesis and increasing colloid pressure, minimize lung edema and cell swelling.

In another embodiment, a method of perfusing a lung is featured. The method includes: positioning the lung in an ex-vivo perfusion circuit; circulating an OCS lung solution specifically for machine perfusion of donor lungs on OCS through the lung, the fluid entering the lung through a pulmonary artery interface and leaving the lung through a left atrial interface; ventilating the lung by flowing a ventilation gas through a tracheal interface; deoxygenating the perfusion solution until a predetermined first value of oxygen content in the perfusion solution is reached; reoxygenating the perfusion solution by ventilating the lung with an oxygenation gas until a predetermined second value of oxygen content in the perfusion solution is reached; and determining a condition of the lung based on a time taken for the lung to cause the oxygen content level in the perfusion solution to change from the first value of oxygen content to the second value of oxygen content.

In another embodiment, a method of producing a solution for perfusing a lung at near physiologic conditions is featured. This method includes combining pre-weighed raw materials including nutrients, colloids, hormones, steroids, buffers and vasodilators with water for injection (“WFI”) and mixed with heating until fully dissolved, monitoring the pH level of the resulting solution, allowing the solution to cool, filtering the cooled solution, dispensing the solution into a primary container and sterilizing the filled container.

In another aspect, a lung care system is featured. The lung system includes: a single use disposable module including an interface adapted to couple the single use disposable module with the multiple use module for electro-mechanical interoperation with the multiple use module; a lung chamber assembly optionally having a first interface for allowing a flow of a lung OCS perfusion solution into the lung, a second interface for allowing ventilation of the lung with a ventilation gas, and a third interface for allowing a flow of the perfusion solution away from the lung, the lung chamber assembly including a dual drain system for carrying the flow of the perfusion solution away from the lung, the dual drain system comprising a measurement drain for directing a part of the perfusion solution flow to a sensor of a perfusion solution gas content and a main drain for receiving a remaining part of perfusion solution flow; and an OCS lung perfusion solution specifically for machine perfusion of donor lungs on OCS.

### BRIEF DESCRIPTION OF THE DRAWINGS

The following figures depict illustrative embodiments in which like reference numerals refer to like elements. These depicted embodiments may not be drawn to scale and are to be understood as being illustrative and not as limiting.

FIG. 1 is a schematic diagram of the lung perfusion circuit of the described embodiment.

FIG. 2 is an illustration of the organ care system drawn from a 45-degree angle from the front view, according to the described embodiment.

FIG. 3 is an illustration of the lung perfusion module, according to the described embodiment.

FIG. 4 is an illustration of the pulmonary artery cannula, according to the described embodiment.

FIG. 5 is an illustration of the tracheal cannula, according to the described embodiment.

FIG. 6 is an exploded illustration of the lung chamber, according to the described embodiment.

FIG. 7 is a schematic diagram of the described embodiment of a portable organ care system including shows the gas-related components of the lung perfusion module.

### DETAILED DESCRIPTION

The following description and the drawings illustrate embodiments sufficiently to enable those skilled in the art to practice them. Other embodiments may incorporate structural, logical, electrical, process, and other changes. Examples merely typify possible variations. Individual components and functions are optional unless explicitly required, and the sequence of operations may vary. Portions and features of some embodiments may be included in or substituted for those of others. The scope of embodiments encompasses the full ambit of the claims and all available equivalents of those claims.

Improved approaches to ex-vivo organ care are provided. More particularly, various embodiments are directed to improved methods and solutions relating to maintaining a lung at or near normal physiologic conditions in an ex-vivo environment. As used herein, “physiological temperature” is referred to as temperatures between about 25 degrees C. and about 37 degrees C. A preferred embodiment comprises a lung OCS perfusion solution that may be administered in conjunction with an organ care system to maintain a lung in an equilibrium state by circulating a perfusion solution through the lung's vascular system, while causing the lung to rebreath a gas having an oxygen content sufficient to met the lung's metabolic needs.

The embodiments allow a lung to be maintained ex-vivo for extended periods of time, such as, for example, 3-24 or more hours. Such extended ex-vivo maintenance times expand the pool of potential recipients for donor lungs, making geographic distance between donors and recipients less important. Extended ex-vivo maintenance times also provide the time needed for better genetic and HLA matching between donor organs and organ recipients, increasing the likelihood of a favorable outcome. The ability to maintain the organ in a near physiologic functioning condition also allows a clinician to evaluate the organ's function ex-vivo, and identify organs that are damaged. This is especially valuable in the case of the lung, since lungs are often compromised as a direct or indirect result of the cause of the death of the donor. Thus even a newly harvested lung may be damaged. The ability to make a prompt assessment of a harvested organ allows a surgeon to determine the quality of a lung and, if there is damage, to make a determination of the nature of the problem. The surgeon can then make a decision as to whether to discard the lung, or to apply therapy to the lung. Therapies can include recruitment processes, removing or stapling off damaged areas of lung, suctioning secretions, cauterizing bleeding blood vessels, and giving radiation treatment. The ability to assess and, if necessary provide therapy to lungs at several stages from harvesting to implantation greatly improves the overall likelihood of lung transplant success and increases the number of organs available for transplant. In some instances, the improved assessment capability and extended maintenance time facilitates medical operators to perform physical repairs on donor organs with minor defects. Increased ex-vivo organ maintenance times can also provide for an organ to be removed from a patient, treated in isolation ex-vivo, and then put back into the body of a patient. Such treatment may include, without limitation, pharmaceutical treatments, gas therapies, surgical treatments, chemo-, bio-, gene and/or radiation therapies.

### Overview of OCS Perfusion Solution

According to certain embodiments, a lung OCS perfusion solution with certain solutes provides for the lungs to function at physiologic or near physiologic conditions and temperature by supplying energy rich nutrients, oxygen delivery, optimal oncotic pressure, pH and organ metabolism. The perfusion solution may also include therapeutic components to help maintain the lungs and protect them against ischemia, reperfusion injury and other ill effects during perfusion. Therapeutics may also help mitigate edema, provide general endothelial tissue support for the lungs, and otherwise provide preventative or prophylactic treatment to the lungs.

The amounts of solutes provided describes preferred amounts relative to other components in the solution and may be scaled to provide compositions of sufficient quantity.

In one embodiment, the solution may include a phosphodiesterase inhibitor. To improve gas exchange and diminish leukocytosis, an adenosine-3′,5′-cyclic monophosphate (cAMP) selective phosphodiesterase type III (PDE III) inhibitor such as milrinone, amrinone, anagrelide, bucladesine, cilostamide, cilostazol, enoximone, KMUP-1, quazinone, RPL-554, siguazodan, trequinsin, vesnarinone, zardaverine may be added. In a preferred embodiment milrinone is added. Milrinone has the effects of vasorelaxation secondary to improved calcium uptake into the sarcoplasmic reticulum, inotropy (myocyte contraction) due to cAMP-mediated trans-sarcolemmal calcium flux, and lusitropy (myocyte relaxation) possibly due to improved actin-myosin complex dissociation. In a preferred embodiment milrinone is present in each 1 L of solution in an amount of about 3400 mcg to about 4600. In a particularly preferred embodiment, milrinone is present in each 1 L of solution in an amount of about 4000 mcg.

In certain embodiments the solution may include a nitrate which is useful in the nitrogen cycle. Nitroglycerin is a nitrate that may be added to the perfusion solution to promote stabilization of pulmonary hemodynamics and improve arterial oxygenation after transplantation. When a lung is removed from the body, nitric oxide levels fall quickly because it is quenched by superoxide generated during reperfusion, resulting in damage to the lung tissue. Nitroglycerin can act to promote nitric oxide levels in a lung ex-vivo by way of intracellular S-nitrosothiol intermediates to directly stimulate guanylate cyclase or to release nitric oxide locally in effector cells. To this end, Nitroglycerin improves vascular homeostasis and improves organ function by providing better arterial oxygenation after transplant. In a preferred embodiment nitroglycerin is present in each 1 L of solution in an amount of about 10 mg to about 50 mg.

In one other embodiment, magnesium sulfate anhydrate may be added to the solution. Pulmonary artery blood pressure is lower than blood pressure in the rest of the body and in the case of pulmonary hypertension, magnesium sulfate promotes vasodilatation in constricted muscles of the pulmonary arteries by modulating calcium uptake, binding and distribution in smooth muscle cells, thereby decreasing the frequency of depolarization of smooth muscle and thus promoting vasodilatation. Magnesium sulfate anhydrate is present in each 1 L of solution in an amount of about 0.083 g to about 0.1127 g. In a particularly preferred embodiment magnesium sulfate anhydrate is present in each 1 L of solution in an amount of about 0.098 g.

In a preferred embodiment, the addition of colloids offers numerous benefits including improving erythrocyte deformability, preventing erythrocyte aggregation, inducing disbanding of already aggregated cells and preserving endothelial-epithelial membrane. Colloids also have anti-thrombotic effects by being able to coat endothelial surfaces and platelets. In this embodiment dextran 40 is present in each 1 L of solution in an amount of about 42.5 g to about 57.5 g. In a particularly preferred embodiment, dextran 40 is present in each 1 L of solution in an amount of about 50 g.

The solution may also contain electrolytes, such as sodium, potassium, chloride, sulfate, magnesium and other inorganic and organic charged species, or combinations thereof. A suitable component may be those where valence and stability permit, in an ionic form, in a protonated or unprotonated form, in salt or free base form, or as ionic or covalent substituents in combination with other components that hydrolyze and make the component available in aqueous solutions. In this embodiment, sodium chloride is present in each 1 L of solution in an amount of about 6.8 g to about 9.2 g. In a particularly preferred embodiment, sodium chloride is present in each 1 L of solution in an amount of about 8 g.

In a preferred embodiment the solution may have a low-potassium concentration. A low-level of potassium results in improved lung function. A low potassium level may also protect the lung during high flow reperfusion and lead to a lower PA pressure and PVR, lower percent decrease in dynamic airway compliance, and lower wet to dry ratio. In this embodiment potassium chloride is present in each 1 L of solution in an amount of about 0.34 g to about 0.46 g. In a particularly preferred embodiment potassium chloride is present in each 1 L of solution in an amount of about 0.4 g.

The solutions may include one or more energy-rich components to assist the organ in conducting its normal physiologic function. These components may include energy rich materials that are metabolizable, and/or components of such materials that an organ can use to synthesize energy sources during perfusion. Exemplary sources of energy-rich molecules include, for example, one or more carbohydrates. Examples of carbohydrates include glucose monohydrate, monosaccharides, disaccharides, oligosaccharides, polysaccharides, or combinations thereof, or precursors or metabolites thereof. While not meant to be limiting, examples of monosaccharides suitable for the solutions include octoses; heptoses; hexoses, such as fructose, allose, altrose, glucose, mannose, gulose, idose, galactose, and talose; pentoses such as ribose, arabinose, xylose, and lyxose; tetroses such as erythrose and threose; and trioses such as glyceraldehyde. In a preferred embodiment glucose monohydrate is present in each 1 L of solution an amount of about 1.7 g to about 2.3 g. In a particularly preferred embodiment glucose monohydrate is present in each 1 L of solution an amount of about 2 g.

The solution may include other components to help maintain the organ and protect it against ischemia, reperfusion injury and other ill effects during perfusion. In certain exemplary embodiments these components may include a hormone to promote and regulate carbohydrate and fat metabolism. Insulin acts to improve cell function by promoting optimum glucose and glycogen intake into the cells. In this preferred embodiment each 1 L of the solution may contain about 17 IU insulin to about 23 IU insulin. In a particularly preferred embodiment each 1 L of the solution may contain 20 IU insulin.

In addition, the solution may include a multi-vitamin that provides anti-oxidants and co-enzymes and helps maintain the body's normal resistance and repair processes. The multi-vitamin may include certain fat soluble vitamins such as Vitamins A, D, E, and K, and water soluble vitamins such as Vitamin C, Niacinamide, Vitamins B₂, B₁, B₆, and Dexpanthenol, as well as stabilizers and preservatives. In a preferred embodiment, each 1 L of the solution contains one unit vial of M.V.I. Adult® multi-vitamin. M.V.I. Adult® includes fat soluble vitamins such as Vitamins A, D, E, and K, and water soluble vitamins such as Vitamin C, Niacinamide, Vitamins B₂, B₁, B₆, and Dexpanthenol, as well as stabilizers and preservatives in an aqueous solution.

The solution may also include an anti-inflammatory agent such as a glucocorticoid steroid. Glucocorticoid steroids act as anti-inflamatory agents by activating to the cell's glucocorticoid receptors which in turn up-regulate the expression of anti-inflammatory proteins in the nucleus and reduce the expression of pro-inflammatory proteins. Glucocorticoid steroids include methylprednisolone, hydrocortisone, cortisone acetate, prednisone, dexamethasone, betamethasone, triamcinolone, beclometasone, fludrocortisone acetate and aldosterone. In this preferred embodiment, each 1 L of the solution may contain about 0.85 g mg to about 1.15 g methylprednisolone (SoluMedrol® or equivalent). In a particularly preferred embodiment, each 1 L of the solution may contain 1 g methylprednisolone (SoluMedrol® or equivalent)

In addition the solution may contain buffers to maintain the solution at an optimal pH. These may include disodium phosphate anhydrate, a physiologic balancing buffer or monopotassium phosphate to maintain the average pH of the solution during lung tissue perfusion. In this embodiment disodium phosphate anhydrate is present in each 1 L of solution in an amount of about 0.039 g to about 0.052 g, and/or monopotassium phosphate in an amount of about 0.053 g to about 0.072 g. In a particularly preferred embodiment, disodium phosphate anhydrate is present in an amount of 0.046 g, and/or monopotassium phosphate in an amount of 0.063 g. In some embodiments, the solution contains sodium bicarbonate, potassium phosphate, or TRIS buffer. In a preferred embodiment the sodium bicarbonate is present in each 1 L of solution in an amount of about 12.75 mEq to about 17.25 mEq. In a particularly preferred embodiment each 1 L of the solution may initially contain about 15 mEq sodium bicarbonate (5 mEq to each 500 mL bottle and 2-3 bottles are used), and additional amounts may be added throughout preservation based on clinical judgment. For example, 20-40 mEq can be added to the system as part of priming.

Other suitable buffers include 2-morpholinoethanesulfonic acid monohydrate (MES), cacodylic acid, H₂CO₃/NaHCO₃ (pKₐ₁), citric acid (pKₐ₃), bis(2-hydroxyethyl)-imino-tris-(hydroxymethyl)-methane (Bis-Tris), N-carbamoylmethylimidino acetic acid (ADA), 3-bis[tris(hydroxymethyl)methylamino]propane (Bis-Tris Propane) (pKₐ₁), piperazine-1,4-bis(2-ethanesulfonic acid) (PIPES), N-(2-Acetamido)-2-aminoethanesulfonic acid (ACES), imidazole, N,N-bis(2-hydroxyethyl)-2-aminoethanesulfonic acid (BES), 3-(N-morpholino)propanesulphonic acid (MOPS), NaH.sub.2PO.sub.4/Na.sub.2HPO.sub.4 (pK.sub.a2), N-tris(hydroxymethyl)methyl-2-aminoethanesulfonic acid (TES), N-(2-hydroxyethyl)-piperazine-N′-2-ethanesulfonic acid (HEPES), N-(2-hydroxyethyl)piperazine-N′-(2-hydroxypropanesulfonic acid) (HEPPSO), triethanolamine, N-[tris(hydroxymethyl)methyl]glycine (Tricine), tris hydroxymethylaminoethane (Tris), glycineamide, N,N-bis(2-hydroxyethyl) glycine (Bicine), glycylglycine (pKₐ₂), N-tris(hydroxymethyl)methyl-3-aminopropanesulfonic acid (TAPS), or a combination thereof.

The solution may contain an antimicrobial or antifungal agent to prevent infection. These may include bacteria and fungal antimicrobial agents that provide protection against both gram negative and gram positive bacteria. Suitable antimicrobial or antifungal agents include cefazolin, ciprofloxacin, and voriconazole or equivalent. In a preferred embodiment, cefazolin is present in each 1 L of solution in an amount of about 0.85 g to about 1.15 g, ciprofloxacin is present in each 1 L of solution in an amount of about 0.17 g to about 2.3 g, and voriconazole is present in each 1 L of solution in an amount of about 0.17 g to about 2.3 g. In a particularly preferred embodiment, cefazolin is present in each 1 L of solution in an amount of about 1 g, ciprofloxacin is present in each 1 L of solution in an amount of about 0.2 g, and voriconazole is present in each 1 L of solution in an amount of about 0.2 g. Alternatively the solution may contain any effective antimicrobial or antifungal agent.

The solutions are preferably provided at a physiological temperature and maintained thereabout throughout perfusion and recirculation.

In a preferred embodiment the OCS lung perfusion solution comprises a nutrient, a colloid, a vasodilator, a hormone and a steroid.

In another preferred embodiment the solution comprises a nutrient including Glucose monohydrate, sodium chloride, potassium chloride, a multi-vitamin including fat-soluble and water-soluble vitamins; a colloid including dextran 40; a hormone including insulin; a steroid including methylprednisolone; buffering agents including disodium phosphate anhydrate, monopotassium phosphate and sodium bicarbonate; vasodilators including milrinone, nitroglycerin and magnesium sulfate anhydrate; antimicrobial or antifungal agents including cefazolin, ciprofloxacin, and voriconazole.

In another preferred embodiment the solution comprises an effective amount of dextran 40; sodium chloride; potassium chloride; magnesium sulfate anhydrate; disodium phosphate anhydrate; monopotassium phosphate; glucose monohydrate; milrinone; nitroglycerin; insulin; a multi-vitamin (M.V.I. Adult® or equivalent); sodium bicarbonate; methylprednisolone (SoluMedrol® or equivalent); cefazolin; ciprofloxacin; voriconazole.

In a preferred embodiment of the OCS lung perfusion solution, each 1 L of solution includes, milrinone in an amount of about 4000 mcg; nitroglycerin in an amount of about 10-50 mg; dextran 40 in an amount of about 50 g; sodium chloride in an amount of about 8 g; potassium chloride in an amount of about 0.4 g; magnesium sulfate anhydrate in an amount of about 0.098 g; disodium phosphate anhydrate in an amount of about 0.046 g; monopotassium phosphate in an amount of about 0.063 g; glucose monohydrate in an amount of about 2 g; insulin in an amount of about 20 IU; a multi-vitamin (M.V.I. Adult® or equivalent) in the amount of about 1 unit vial; sodium bicarbonate is initially present in an amount of about 15 mEq; methylprednisolone in an amount of about 1 g.

In a particularly preferred embodiment of the OCS lung perfusion solution, each 1 L of solution includes, milrinone in an amount of about 4000 mcg; nitroglycerin in an amount of about 10-50 mg; dextran 40 in an amount of about 50g; sodium chloride in an amount of about 8 g; potassium chloride in an amount of about 0.4 g; magnesium sulfate anhydrate in an amount of about 0.098 g; disodium phosphate anhydrate in an amount of about 0.046 g; monopotassium phosphate in an amount of about 0.063 g; glucose monohydrate in an amount of about 2 g; insulin in an amount of about 20 IU; a multi-vitamin (M.V.I. Adult® or equivalent) in the amount of about 1 unit vial; sodium bicarbonate is initially present in an amount of about 15 mEq; methylprednisolone in an amount of about 1 g; cefazolin in an amount of about 1 g; ciprofloxacin in an amount of about 0.2 g; voriconazole in an amount of about 0.2 g.

In certain embodiments, the perfusion solution is maintained and provided to the lungs at a near physiologic temperature. According to one embodiment, the perfusion solution employs a blood product-based perfusion solution to more accurately mimic normal physiologic conditions. The perfusion solution may be supplemented with cellular media. The cellular media may include a blood product, such as whole blood, or packed red blood cells; allogenic packed red blood cells that are leukocyte depleted/reduced; donor's whole blood that is leukocyte and platelet depleted/reduced; and/or human plasma to achieve circulating hematocrit of 15-30%.

### Overview of Method of Producing a Solution for Perfusing a Lung at Near Physiologic Temperature

In another aspect, a method of producing a solution for perfusing a lung at near physiologic temperature is provided. In a preferred method, the pre-weighed raw materials and WFI are added to a stainless steel mixing tank and mixed with heating until fully dissolved. The pH of the resulting solution is monitored and adjusted during the mixing process with 1M hydrochloric acid (HCl). The solution is allowed to cool and then filtered through a 0.2 μm filter and finally dispensed into a primary container. The filled container is terminally sterilized with heat using a sterilization cycle that has been validated to achieve a Sterility Assurance Level of 10⁻⁶. The raw materials in a preferred embodiment include a nutrient, a colloid, a vasodilator, a hormone and a steroid for perfusing a lung at near physiologic conditions.

In another preferred embodiment the raw materials include a nutrient including glucose monohydrate, sodium chloride, potassium chloride, a multi-vitamin including M.V.I. Adult® or equivalent; a colloid including dextran 40; a hormone including insulin; a steroid including methylprednisolone; buffering agents including disodium phosphate anhydrate, monopotassium phosphate and sodium bicarbonate; vasodilators including milrinone, nitroglycerin and magnesium sulfate anhydrate; an antimicrobial or antifungal agent.

In another preferred embodiment the raw materials include dextran 40; sodium chloride; potassium chloride; magnesium sulfate anhydrate; disodium phosphate anhydrate; monopotassium phosphate; glucose monohydrate; milrinone; nitroglycerin; insulin; a multi-vitamin (M.V.I. Adult® or equivalent); sodium bicarbonate; methylprednisolone (SoluMedrol® or equivalent); antimicrobial or antifungal agents including cefazolin, ciprofloxacin, and voriconazole for perfusing a lung at near physiologic conditions.

In a preferred embodiment, for each 1 L of solution, the raw materials include milrinone in an amount of about 4000 mcg; nitroglycerin in an amount of about 10-50 mg; dextran 40 in an amount of about 50 g; sodium chloride in an amount of about 8 g; potassium chloride in an amount of about 0.4 g; magnesium sulfate anhydrate in an amount of about 0.098 g; disodium phosphate anhydrate in an amount of about 0.046 g; monopotassium phosphate in an amount of about 0.063 g; glucose monohydrate in an amount of about 2 g; insulin in an amount of about 20 IU; a multi-vitamin (M.V.I. Adult® or equivalent) in the amount of about 1 unit vial; sodium bicarbonate is initially present in an amount of about 15 mEq; methylprednisolone in an amount of about 1 g; an antimicrobial or antifungal agent.

In another particularly preferred embodiment, for each 1 L of solution, the raw materials include milrinone in an amount of about 4000 mcg; nitroglycerin in an amount of about 10-50 mg; dextran 40 in an amount of about 50 g; sodium chloride in an amount of about 8 g; potassium chloride in an amount of about 0.4 g; magnesium sulfate anhydrate in an amount of about 0.098 g; disodium phosphate anhydrate in an amount of about 0.046 g; monopotassium phosphate in an amount of about 0.063 g; glucose monohydrate in an amount of about 2 g; insulin in an amount of about 20 IU; a multi-vitamin (M.V.I. Adult® or equivalent) in the amount of about 1 unit vial; sodium bicarbonate is initially present in an amount of about 15 mEq; methylprednisolone in an amount of about 1 g; cefazolin in an amount of about 1 g; ciprofloxacin in an amount of about 0.2 g; voriconazole in an amount of about 0.2 g.

### Overview of Method of Flushing an Organ with a Solution Between Excise from the Donor and Instrumentation on OCS

In another aspect, there is provided a method of flushing an organ with a solution between excise from the body and instrumentation on OCS. In this embodiment, to prepare a donor lung for surgical removal from the donor's chest and to remove all old donor blood from the lung, the donor lung is flushed ante-grade using the pulmonary artery with the solution until the temperature of the donor lung is in the range of about 0 degrees C. to about 30 degrees C. Additionally, the solution may be used for retrograde flush of the lung using the pulmonary veins to remove any blood clots remaining in the donor lung prior to surgical removal of the lung from the donor's chest, and to ensure adequate homogenous distribution of flush solution to all lung segments. The lungs are ventilated using a ventilator during both ante-grade and retro-grade flushing to allow for homogenous distribution of the solution and to increase the oxygen concentration in the donor lung alveoli to minimize the impact of ischemia/reperfusion injury on the donor lung. Once the ante-grade and retrograde flushing of the donor lung is completed, the lung will be removed surgically while inflated to minimize collapsing of the alveoli. Once the donor lung is fully removed from the donor body, it is ready to the next phase of OCS perfusion.

In one embodiment, the solution comprises an energy-rich perfusion nutrient, a colloid, a hormone, a buffer, magnesium sulfate anhydrate, and a nitrate. In another embodiment, the solution comprises dextran 40; sodium chloride; potassium chloride; magnesium sulfate anhydrate; disodium phosphate anhydrate; monopotassium phosphate; glucose monohydrate; nitroglycerin.

In a particularly preferred embodiment each 1 L of solution for ante-grade flush comprises dextran 40 in an amount of about 50 g; sodium chloride in an amount of about 8 g; potassium chloride in an amount of about 0.4 g; magnesium sulfate anhydrate in an amount of about 0.098 g; disodium phosphate anhydrate in an amount of about 0.046 g; monopotassium phosphate in an amount of about 0.063 g; glucose monohydrate in an amount of about 2 g; nitroglycerin in an amount of about 50 mg.

In another particularly preferred embodiment each 1 L of solution for retrograde flush comprises dextran 40 in an amount of about 50 g; sodium chloride in an amount of about 8 g; potassium chloride in an amount of about 0.4 g; magnesium sulfate anhydrate in an amount of about 0.098 g; disodium phosphate anhydrate in an amount of about 0.046 g; monopotassium phosphate in an amount of about 0.063 g; glucose monohydrate in an amount of about 2 g; nitroglycerin in an amount of about 10 mg.

### Overview of Method of Machine Perfusion Using Lung OCS Perfusion Solution

In another aspect, a method for machine perfusion of a donor lung is provided. The method includes perfusing the donor lung with a OCS lung perfusion solution comprising: dextran 40; sodium chloride; potassium chloride; magnesium sulfate anhydrate; disodium phosphate anhydrate; monopotassium phosphate; glucose monohydrate; milrinone; nitroglycerin; insulin; at least two vitamins; sodium bicarbonate; methylprednisolone (SoluMedrol® or equivalent); a microbial or antifungal agent.

In a further aspect, the method includes perfusing the donor lung with a particularly preferred OCS lung perfusion solution comprising for each 1 L of solution: milrinone in an amount of about 4000 mcg; nitroglycerin in an amount of about 10-50 mg; dextran 40 in an amount of about 50 g; sodium chloride in an amount of about 8 g; potassium chloride in an amount of about 0.4 g; magnesium sulfate anhydrate in an amount of about 0.098 g; disodium phosphate anhydrate in an amount of about 0.046 g; monopotassium phosphate in an amount of about 0.063 g; glucose monohydrate in an amount of about 2 g; insulin in an amount of about 20 IU; a multi-vitamin (M.V.I. Adult® or equivalent) in the amount of about 1 unit vial; sodium bicarbonate is initially present in an amount of about 15 mEq; methylprednisolone in an amount of about 1 g; cefazolin in an amount of about 1 g; ciprofloxacin in an amount of about 0.2 g; voriconazole in an amount of about 0.2 g.

### Overview of the Lung Perfusion Circuit

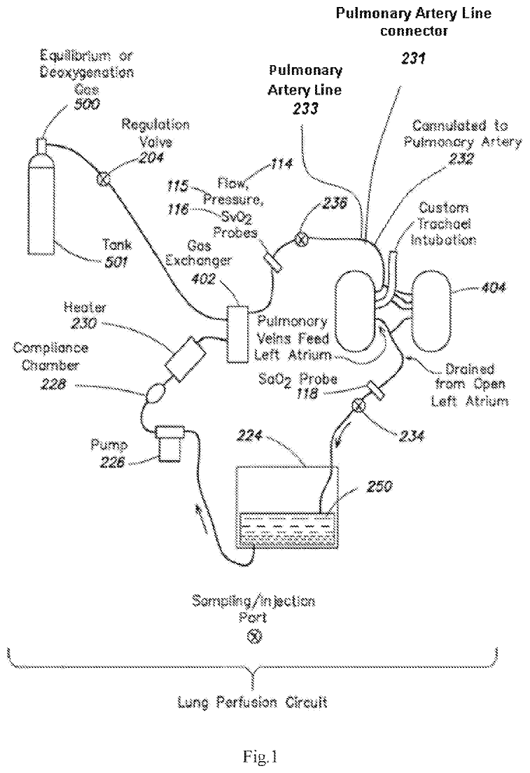

FIG. 1 illustrates an exemplary lung perfusion circuit which can be used to circulate the perfusion solution noted above. The circuit is housed entirely within a lung perfusion module, and all its components may be disposable. The organ care system (OCS) disclosure, U.S. application Ser. No. 12/099,715, includes an exemplary embodiment of a lung perfusion circuit and is incorporated in its entirety by reference. Lung OCS perfusion solution 250 is placed in a reservoir and then circulates within the perfusion circuit, passing through various components of lung perfusion module before passing through the vascular system of lungs 404. Pump 226 causes perfusion solution 250 to flow around the lung perfusion circuit. It receives perfusion solution 250 from reservoir 224, and pumps the solution through compliance chamber 228 to heater 230. Compliance chamber 228 is a flexible portion of tubing that serves to refine the flow characteristics nature of pump 226. Heater 230 replaces heat lost by perfusion solution 250 to the environment during circulation of the fluid. In the described embodiment, the heater maintains perfusion solution 250 at or near the physiologic temperature of 30-37 degrees C., and preferably at about 34 degrees C. After passing through heater 230, perfusion solution 250 flows into gas exchanger 402. Gas exchanger 402 allows gases to be exchanged between gas and perfusion solution 250 via a gas-permeable, hollow fiber membrane. However, the gas exchanger has an effective gas exchange surface area of about 1 square meter, which is only a fraction of the 50-100 square meter effective exchange area of the lungs. Thus gas exchanger 402 has only a limited gas exchange capability compared to the lungs. Blood gas solenoid valve 204 regulates the supply of gas into gas exchanger 402. The composition of gas supplied to gas exchanger is determined by which mode the OCS is in. For example, when OCS 100 is in a sequential assessment mode, deoxygenation gas 500 from deoxygenation gas tank 501 is supplied to the gas exchanger. Sampling/injection port 236 facilitates the removal of a sample or the injection of a chemical just before perfusion solution 250 reaches the lungs. Perfusion solution then enters lungs 404 through cannulated pulmonary artery 232. Flow probe 114 measures the rate of flow of perfusion fluid 250 through the system. In the described embodiment, flow probe 114 is placed on the perfusate line as it leads towards the pulmonary artery. Pressure sensor 115 measures pulmonary arterial pressure at the point of entry of perfusion fluid 250 into the lungs. Oxygen probe 116 measures oxygen in perfusion fluid 250 just before it enters the lungs. In the described embodiment, perfusion solution 250 is the lung OCS solution described previously.

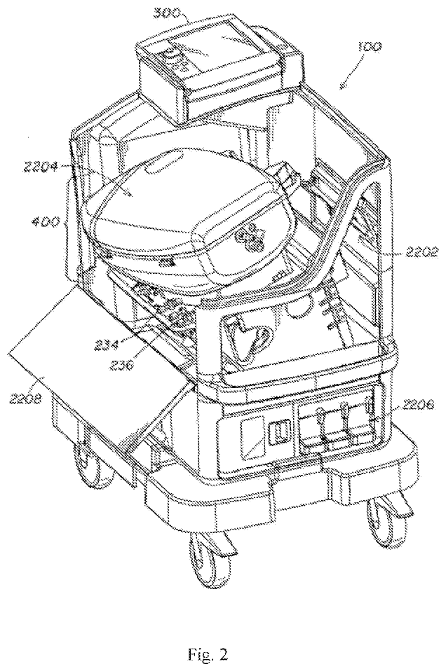

FIG. 2 is an overall view of OCS console 100 showing the single use, disposable lung perfusion module in a semi-installed position. As broadly indicated in FIG. 2, single use disposable lung perfusion module is sized and shaped to fit into OCS console 100, and to couple with it. Overall, the unit has a similar form to the organ care system described in U.S. patent application Ser. No. 11/788,865. Removable lung perfusion module 400, is insertable into OCS console 100 by means of a pivoting mechanism that allows module 400 to slide into the organ console module from the front, as shown in FIG. 2, and then pivot towards the rear of the unit. Clasp mechanism 2202 secures lung perfusion module 400 in place. In alternative embodiments, other structures and interfaces of lung perfusion module 400 are used to couple the module with OCS 100. When secured in place, electrical and optical connections (not shown) provide power and communication between OCS console 100 and lung perfusion module 400. Details of the electrical and optical connections are described in U.S. patent application Ser. No. 11/246,013, filed on Oct. 7, 2005, the specification of which is incorporated by reference herein in its entirety. A key component of lung perfusion module 400 is organ chamber 2204, which is described in detail below. Battery compartments 2206 and maintenance gas cylinder 220 (not shown) are located in the base of the OCS console 100. OCS console 100 is protected by removable panels, such as front panels 2208. Just below lung perfusion module are perfusion solution sampling ports 234 and 236. Mounted on top of OCS console 100 is OCS monitor 300.

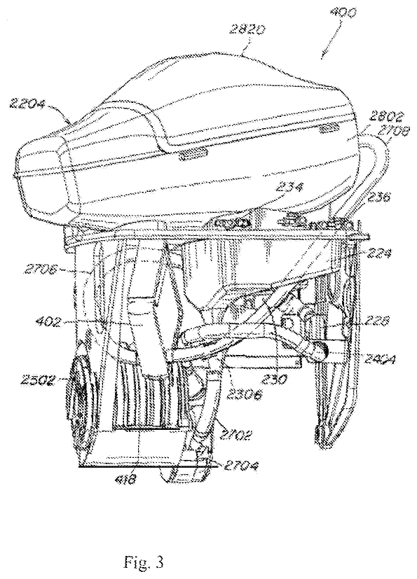

FIG. 3 is a front view of lung perfusion module 400. Organ chamber 2204 includes a removable lid 2820 and housing 2802. Sampling ports, including LA sampling port 234 and PA sampling port 236 are visible below organ chamber 2802. Gas exchanger 402, bellows 418, and bellows plate 2502 are also visible in the figure.

The circulation path of the perfusion solution, which was first described in connection with FIG. 2, in terms of the components of lung perfusion module 400 is now addressed. Mounted below organ chamber 2204 are perfusion solution reservoir 224, which stores perfusion solution 250. The perfusion solution exits through one-way inflow valve 2306, line 2702, and pump dome 2704 to pump 226 (not shown). The perfusion solution is pumped through perfusion solution line 2404 through compliance chamber 228, and then to perfusion solution heater 230. After passing through heater 230, the perfusion solution passes through connecting line 2706 to gas exchanger 402.

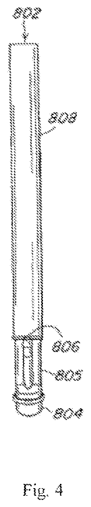

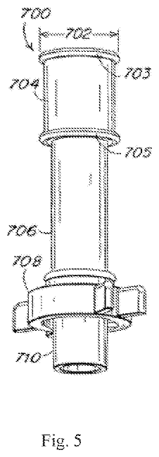

The pulmonary artery (PA) cannula connects the perfusion circuit with the vascular system of lungs 404. An exemplary embodiment of a pulmonary artery (PA) cannula is shown in FIG. 4. Referring to FIG. 4, single PA cannula 802 has single insertion tube 804 for insertion into a single PA, and is used to cannulate the PA at a point before it branches to the two lungs. To connect the cannula to the pulmonary artery, insertion tube 804 is inserted into the PA, and the PA is secured onto the tube with sutures. The tracheal cannula 700 is inserted into the trachea to provide a means of connection between the lung perfusion module 400 gas circuit and the lungs. FIG. 5 illustrate an exemplary tracheal cannulae. Cannula 700 includes tracheal insertion portion 704 having an insertion portion tip diameter 702, to which the trachea is secured with a cable tie, or by other means. At the end of insertion portion 704 that is inserted into the trachea is rib 703; the rib helps secure insertion portion 704 at the inserted location within the trachea, and is secured with a cable tie placed around the trachea. At the opposite end of insertion portion 704, second rib 705, having a diameter about 0.2 inches greater than the base part diameter of insertion portion 704, acts as a stop for the silicone over-layer and as a stop for the trachea. The tracheal cannula may be clamped at flexible portion 706 prior to instrumentation to seal off air flow in and out of the lungs 404. Also illustrated is an optional locking nut 708.

The perfusion solution exits gas exchanger 402 through connecting line 2708 to the interface with the pulmonary artery. After flowing through the lung and exiting via the pulmonary vein and the left atrium, the perfusion solution drains through from the base of organ chamber 2204, as described below. These drains feed the perfusion solution to reservoir 224, where the cycle begins again.

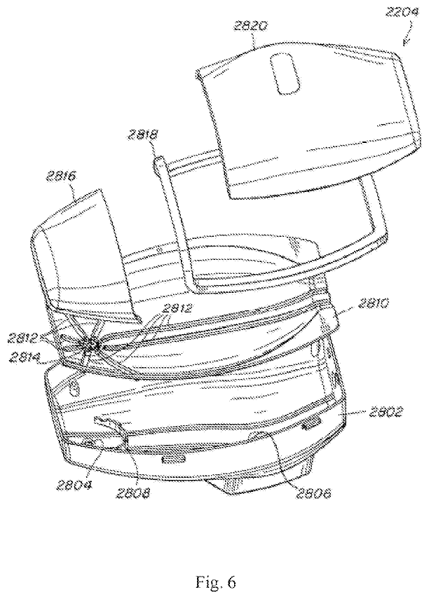

Having described OCS console 100 and lung perfusion module 400, we now describe organ chamber 2204. FIG. 6 shows an exploded view of the components of organ chamber 2204. The top of organ chamber 2204 is covered with a sealable lid that includes front piece 2816, top piece 2820, inner lid with sterile drape (not shown), and sealing piece 2818 that seals front piece 2816 to top piece 2820. Base 2802 of chamber 2204 is shaped and positioned within lung perfusion module 400 to facilitate the drainage of the perfusion solution. Organ chamber 2204 has two drains, measurement drain 2804, and main drain 2806, which receives overflow from the measurement drain. Measurement drain 2804 drains perfusion solution at a rate of about 0.5 l/min, considerably less than perfusion solution 250 flow rate through lungs 404 of between 1.5 l/min and 4 l/min. Measurement drain leads to oxygen probe 118, which measures SaO₂ values, and then leads on to reservoir 224. Main drain 2806 leads directly to reservoir 224 without oxygen measurement. Oxygen probe 118, which is a pulse oxymeter in the described embodiment, cannot obtain an accurate measurement of perfusion solution oxygen levels unless perfusion solution 250 is substantially free of air bubbles. In order to achieve a bubble-free column of perfusion solution, base 2802 is shaped to collect perfusion solution 250 draining from lungs 404 into a pool that collects above drain 2804. The perfusion solution pool allows air bubbles to dissipate before the perfusion solution enters drain 2804. The formation of a pool above drain 2804 is promoted by wall 2808, which partially blocks the flow of perfusion solution from measurement drain 2804 to main drain 2806 until the perfusion solution pool is large enough to ensure the dissipation of bubbles from the flow. Main drain 2806 is lower than measurement drain 2804, so once perfusion solution overflows the depression surrounding drain 2804, it flows around wall 2808, to drain from main drain 2806. In an alternate embodiment of the dual drain system, other systems are used to collect perfusion solution into a pool that feeds the measurement drain. In some embodiments, the flow from the lungs is directed to a vessel, such as a small cup, which feeds the measurement drain. The cup fills with perfusion solution, and excess blood overflows the cup and is directed to the main drain and thus to the reservoir pool. In this embodiment, the cup performs a function similar to that of wall 2808 in the embodiment described above by forming a small pool of perfusion solution from which bubbles can dissipate before the perfusion solution flows into the measurement drain on its way to the oxygen sensor.

Lungs 404 are supported by support surface 2810. The surface is designed to support lungs 404 without applying undue pressure, while angling lungs 404 slightly downwards towards the lower lobes to promote easy drainage of the perfusion solution. Support surface includes drainage channels 2812 to collect and channel perfusion solution issuing from lungs 404, and to guide the perfusion solution towards drain 2814, which feeds perfusion solution directly to the blood pool for measurement drain 2804. To provide additional support for the lungs, lungs 404 are wrapped with a polyurethane wrap (not shown) when placed on support surface 2810. The polyurethane wrap anchors lungs 404, helps keep the lungs in a physiologic configuration, and prevents the bronchi from being kinked and limiting the total volume of inflation. The wrap provides a smooth surface for the exterior of the lung to interface with organ chamber 2204, reducing the risk of the chamber applying excessive pressure on any part of lungs 404, which might cause undesirable hemorrhaging.

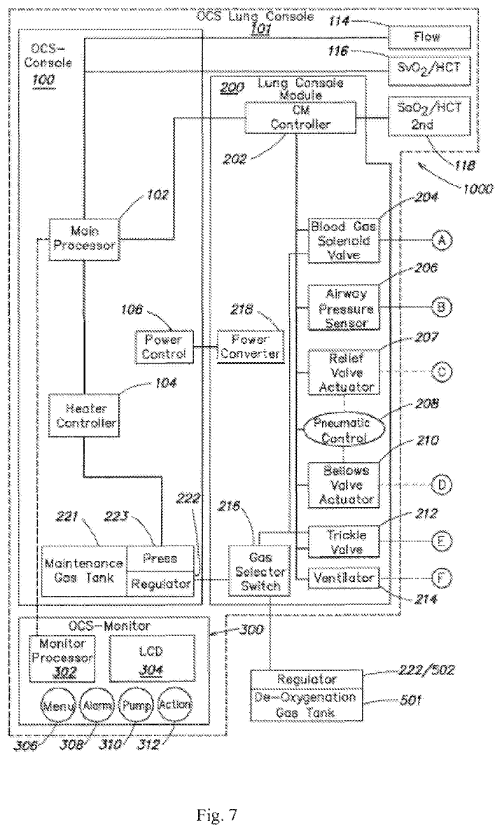

FIG. 7 is a schematic diagram of the described embodiment of a portable organ care system including the gas-related components of the lung perfusion module. The organ care system 1000 includes a permanent, multiple use, non-disposable section, OCS lung console 101, and a single use disposable section, lung perfusion module 400. Regulator 222/502 converts the gas tank pressure to 25 mm Hg for use in the system. Internal maintenance gas tank 221 contains a mixture that is designed to provide enough oxygen to maintain the lung tissue during maintenance mode. Pressure transducer 223 measures the pressure of internal maintenance gas in tank 221, so that the amount of gas remaining can be determined. Controller 202 manages the release of maintenance and assessment gases by controlling the valves, gas selector switch 216, and ventilator 214, thus implementing the preservation of the lungs in maintenance mode, or the assessment of the lungs in one of the assessment modes. Blood gas solenoid valve 204 controls the amount of gas flowing into blood gas exchanger 402. Airway pressure sensor 206 samples pressure in the airway of lungs 404, as sensed through isolation membrane 408. Relief valve actuator 207 is pneumatically controlled, and controls relief valve 412. The pneumatic control is carried out by inflating or deflating orifice restrictors that block or unblock the air pathway being controlled. This method of control allows complete isolation between the control systems in lung console module 200 and the ventilation gas loop in lung perfusion module 400. Pneumatic control 208 controls relief valve 207 and bellows valve actuator 210. Trickle valve 212 controls delivery of gas to the airway of lungs 404. Ventilator 214 is a mechanical device with an actuator arm that causes bellows 418 to contract and expand, which causes inhalation and exhalation of gas into and out of lungs 404. OCS monitor 300 provides user control of OCS 1000 via buttons, and displays data from the system's sensors that indicate the state of the lungs and of the various subsystems within OCS 1000. Monitor 300 is universal, i.e., it can be used for any organ. It includes monitor processor 302 that runs the software controlling monitor 300 and displays data on LCD 304. OCS monitor 300 includes four control buttons for the user: menu button 306 brings up the configuration menu; alarm button 308 silences the speaker; pump button 310 controls the circulatory pump; and action button 312 provides access to certain organ-specific actions, such as ventilator control, or to system actions, such as saving a session file to an external memory card. Other controls can also be included, such as a knob for controlling a value or selecting an item.

### Use Models

An exemplary model for using the solution described above in the organ care system is described below.

The process of preparing the OCS perfusion module 400 for instrumentation begins by producing the solution by the method of producing a solution for perfusing a lung at near physiologic temperature as described previously. About 800 ml to about 2000 ml of the OCS lung perfusion solution is then added into the Organ Care System (OCS) sterile perfusion module 400. The solution is then supplemented with about 500 ml to about 1000 ml of cellular media. The cellular media may include one or combination of the following to achieve total circulating hematocrit concentration between 15-30%: typed allogenic packed red blood cells (pRBCs) that is leukocytes depleted/reduce; donor's whole blood that is leukocyte and platelet depleted/reduced; and/or human plasma to achieve circulating hematocrit of 15-30%. The OCS device operates to circulate and mix the solution and cellular media while warming and oxygenating the solution using a built in fluid warmer and gas exchanger 402. Once the solution is fully mixed, warmed and oxygenated, the pH of the solution will be adjusted using sodium bicarbonate or other available buffer solution as needed. Once the solution's hematocrit, temperature and pH levels reach an acceptable state, the donor lung will be instrumented on OCS.

Once the solution is fully mixed, pH is adjusted to 7.35-7.45 and hematocrit is adjusted to 15-30%, the donor lung will be instrumented on OCS. To begin instrumentation, first set the flow rate of the OCS Pump 226 to about 0.05 L/min. to ensure that perfusion solution does not exit the PA line 233 prior to connecting the trachea cannula 700. Place the lung in the OCS' organ chamber 224 and connect the trachea cannula 700 to the OCS trachea connector 710 and unclamp trachea cannula at section 706. Then connect a PA pressure monitoring line with pressure sensor 115, to the PA cannula 802, including pressure transducer connector 806. To connect the cannula to the pulmonary artery, insertion tube 804 is inserted into the PA, and the PA is secured onto the tube with sutures. Insertion tube 804 of cannula 802 connects to connector portion 805, which serves to position insertion tube 804 at an angle and location suitable for strain-free connection to the pulmonary artery of lungs 404. Connection portion 805 connects to main tube portion 808, which is attached to the perfusion fluid circuit. Trim the OCS' PA cannula 802 and prepare to connect to the OCS PA line connector 231. Next, increase the OCS' pump 226 flow to about 0.3 to about 0.4 L/min. so that a low-flow column of solution exits the PA line 233. Then remove any air from the lung by connecting the lung PA cannula 802 to the OCS PA line connector 231 and gradually filling the PA cannula 802 with perfusion solution. Once an air-free column of solution is reached inside the PA cannula 802, seal the connection between the PA cannula 802 and the OCS PA line connector 231.

Next, gradually raise the OCS fluid warmer 230 temperature to 37 degrees C., and bring the perfusion solution temperature from about 32 degrees C. to about 37 degrees C. Then begin increasing the pump flow gradually, ensuring that pulmonary arterial pressure (“PAP”) remains below 20 mmHg, until pulmonary flow rate reaches a target flow rate of at least 1.5 L/min. When the lung reaches a temperature of about 30 degrees C. to about 32 degrees C., begin OCS ventilation by turning the OCS ventilator 214 to “preservation” mode. The ventilator settings for instrumentation and preservation are specified in Table 1.

TABLE 1
Ventilator Settings (Instrumentation and Preservation)

| Parameter | Requirement |
|---|---|
| Tidal Volume (TV) | = or <6 ml/kg |
| Respiratory Rate (RR) | 10 breaths/min |
| Positive End Expiratory | 7-8 cm H₂O |
| Pressure (PEEP) | Note: decrease to 5 cmH₂O after confirming |
| | adequate inflation of lungs (within 2 hours) |
| I:E Ratio | 1:2-1:3 |
| Peak Airway Pressure | <25 cmH2O |
| (PAWP) | |

Next, gradually increase the perfusion and ventilation rate for up to about 30 minutes until reaching full ventilation and perfusion and allow ventilation parameters to stabilize. Once ventilation parameters of the donor lung on OCS have stabilized, wrap the lung to avoid over inflation injury to the donor lung ex-vivo. The lung may also be wrapped during “pause preservation” before beginning ventilation. During preservation of lung on OCS, ventilation settings are maintained as described in Table 1, the mean PAP is maintained under about 20 mmHg, and the pump flow is maintained at not less than about 1.5 L/min. Blood glucose, electrolytes and pH levels are monitored and adjusted within normal physiologic ranges by additional injections. Lung oxygenation function may be assessed using the OCS lung system in addition to lung compliance. In some instances it is desirable to provide therapy to the lung as described previously. Fiberoptic bronchoscopy may be performed for the donor lung ex-vivo on the OCS device. Once preservation and assessment of the donor lung on the OCS system is complete, the lung is cooled and removed from the OCS system to be transplanted into the recipient.

Donor lung cooling may be achieved by first shutting off the OCS pulsatile pump 226 and flush the donor lung with about 3 liters of perfusion solution at a temperature of about 0 degrees C. to about 15 degrees C. while continuing ventilation on the OCS system. Once the flush is complete the trachea 700 and pulmonary artery 802 cannulae may be disconnected from the OCS and the lung will be immersed in cold preservation solution until it is surgically attached to the recipient (transplanted). Alternatively, the entire system circulating OCS solution may be cooled down to 0 degrees C. to about 15 degrees C. using a heat-exchanger and cooling device while the lung is being ventilated on OCS. Once the target temperature of about 0 degrees C. to about 15 is achieved, the trachea 700 and pulmonary artery 802 cannulae will be disconnected from the OCS and the lung will be immersed in cold preservation solution until it is surgically attached to the recipient (transplanted).

The described system may utilize any embodiment of the lung OCS perfusion solution. In a preferred embodiment, the solution is mixed with red blood cells and placed into a system reservoir for use in the system.

It is to be understood that while the invention has been described in conjunction with the various illustrative embodiments, the forgoing description is intended to illustrate and not limit the scope of the invention, which is defined by the scope of the appended claims. For example, a variety of systems and/or methods may be implemented based on the disclosure and still fall within the scope of the invention. Other aspects, advantages, and modifications are within the scope of the following claims. All references cited herein are incorporated by reference in their entirety and made part of this application.

1. A system for perfusing a donor lung in a lung perfusion circuit at or near physiologic conditions comprising:
a single use disposable lung care module including an interface adapted for attachment to the single use module,
a lung chamber assembly having a first interface for allowing a flow of a perfusion solution into the lung and a second interface for allowing ventilation of the lung with a ventilation gas, and
a drain system for draining a flow of perfusion solution from the lung chamber assembly,
wherein the perfusion solution includes dextran 40; sodium chloride; potassium chloride; magnesium sulfate anhydrate; disodium phosphate anhydrate; monopotassium phosphate; glucose monohydrate; milrinone; nitroglycerin; insulin; a multi-vitamin; sodium bicarbonate; and methylprednisolone.

2. The system of claim 1, further comprising a heater for replacing heat lost by the perfusion solution during circulation of the perfusion solution through the lung perfusion circuit.

3. The system of claim 2, wherein the heater maintains the perfusion solution between temperatures of 30° C. and 37° C.

4. The system of claim 1, further comprising a pulmonary artery cannula that connects the lung perfusion circuit with a vascular system of the lung.

5. The system of claim 4, wherein the pulmonary artery cannula includes an insertion tube for insertion into a pulmonary artery of the lung.

6. The system of claim 1, wherein the lung chamber assembly includes a housing, a support surface, and a removable lid.

7. The system of claim 6, wherein the support surface comprises a wrap configured to support the lung.

8. The system of claim 7, wherein the wrap comprises polyurethane.

9. The system of claim 1, wherein the ventilation gas is selectable from a maintenance gas and an assessment gas.

10. The system of claim 1, further comprising a bellows, that causes the ventilation gas to enter and exit the lung.

11. The system of claim 10, wherein the bellows is controlled by a mechanical ventilator with an actuator arm.

12. The system of claim 1, wherein a trickle valve controls supply of the ventilation gas to maintain a predetermined composition of the ventilation gas.

13. The system of claim 1, wherein the second interface includes a tracheal cannula.

14. The system of claim 13, wherein the tracheal cannula further comprises
a tracheal insertion portion for inserting into the trachea, and
a flexible portion.

15. The system of claim 14, wherein the flexible portion can be clamped to seal off ventilation gas flow in and out of the lung.

16. The system of claim 14, wherein the flexible portion further comprises a locking nut for securing the tracheal cannula to the lung chamber assembly.

17. The system of claim 7, wherein the wrap is further configured to anchor the lung.
